(19) United States
(12) Patent Application Publication
(10) Pub. No.: US 2024/0412870 A1
(43) Pub. Date: Dec. 12, 2024

(54) SYSTEM AND METHOD FOR MEDICAL PROTOCOL AND DISPOSITION

(71) Applicant: Charu Software Solutions, LLC, Jacksonville, FL (US)

(72) Inventors: Ravi K. Raheja, Nashville, TN (US); Charu G. Raheja, Winston-Salem, NC (US)

(21) Appl. No.: 18/736,276

(22) Filed: Jun. 6, 2024

Related U.S. Application Data
(60) Provisional application No. 63/506,479, filed on Jun. 6, 2023.

(51) Int. Cl. G16H 50/20; G16H 10/20; G16H 10/60; G16H 15/00; G16H 20/00; G16H 50/50; G16H 80/00

(52) U.S. Cl. CPC G16H 50/20; G16H 10/20; G16H 10/60; G16H 15/00; G16H 20/00; G16H 50/50; G16H 80/00

(57) ABSTRACT

Systems and methods are disclosed to improve the efficiency and accuracy of capturing and analyzing medical symptoms relayed to medical providers. A medical assistance system and method may be used to provide intelligent medical scripting and analysis, and may improve the ability of providers to make more accurate clinical decisions. A history assistance device may ensure that all relevant symptoms have been asked. Protocol assistance system and method may provide guidance to improve selection of a medical protocol (e.g., select a best protocol based on a decision tree), where the medical protocol is used to assess the patient symptoms and determine an appropriate medical disposition. A safety assistance system and method may use data generated by the medical assistance and protocol assistance to determine recommended level of care for the patient based on the patient medical symptoms, severity, and duration, and to track and analyze any deviations from the provider's assessment and the recommended medical disposition.

### CROSS-REFERENCE TO RELATED APPLICATION

This patent application claims the benefit of U.S. Provisional Patent Application No. 63/506,479, filed Jun. 6, 2023, entitled “SYSTEM AND METHOD FOR MEDICAL ASSISTANCE ANALYSIS”, which is incorporated by reference herein in its entirety.

### TECHNICAL FIELD

Embodiments described herein generally relate to communicating and analysis of medical information.

### BACKGROUND

Communication between a medical patient and medical service provider may face several obstacles. When a patient experiences a medical symptom, the patient may contact a telehealth provider or in-person medical services provider, and a receptionist or answering service may relay the message to a medical service provider. However, the communication between the patient and receptionist or answering service may not correctly diagnose or identify the severity the medical symptom.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 is a flowchart illustrating a medical communication and triage assistant, according to an embodiment.

FIG. 2 is a graphical user interface (GUI) diagram illustrating an introductory display, according to an embodiment.

FIG. 3 shows a GUI displaying prompts to the medical practitioner, according to an embodiment.

FIG. 4 shows additional methods of entering symptoms into the GUI, according to an embodiment.

FIG. 5 better shows possible protocol selections, according to an embodiment.

FIG. 6 is a graphical user interface diagram, according to an embodiment.

FIG. 7 is a block diagram of a computing device method, according to an embodiment.

FIG. 8 is a block diagram of a computing device, according to an embodiment.

### DETAILED DESCRIPTION

Systems and methods are disclosed to improve the efficiency and accuracy of capturing and analyzing medical information relayed to medical providers. A medical assistance system and method may be used to provide intelligent medical scripting and analysis, and may improve the ability of nonclinical operators take more accurate medical messages. A history assistance system and method may provide medical triage providers with a quick guidance to ask the most relevant questions quickly and efficiently. A protocol assistance system and method may provide a medical triage algorithm to assess patient symptoms and determine an appropriate medical disposition. A safety assistance system and method may use data generated by the medical assistance and protocol assistance to determine patient medical issue severity and duration, and to track and analyze any deviations from a recommended medical disposition.

In the following description, for purposes of explanation, numerous specific details are set forth to provide a thorough understanding of some example embodiments. It will be evident, however, to one skilled in the art that the present disclosure may be practiced without these specific details.

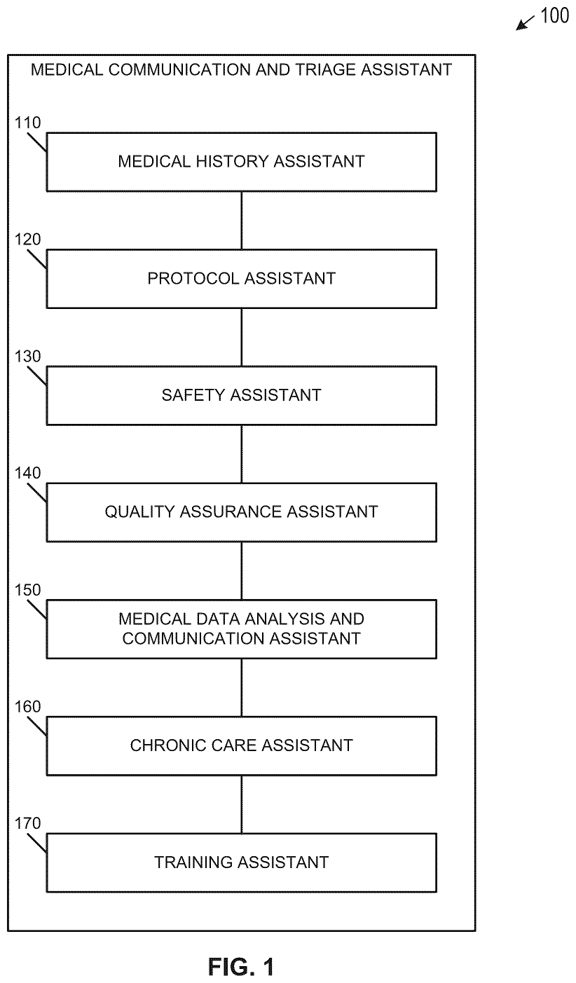

FIG. 1 is a flowchart illustrating a medical communication and triage assistant 100, according to an embodiment. The flowchart illustrates different steps into generating a disposition recommendation, starting with the input of medical history, using the medical history assistant, which prompts the medical practitioner to inquire about different medical history that may be pertinent to determining a proper disposition. The flowchart next illustrates the protocol assistant step, where the application prompts the medical practitioner to inquire about different symptoms the patient has present, along with other factors of the symptoms including severity, duration, and other medical data. The flowchart continues onto the safety assistance step where the application compares the generated result with the conclusion of the medical practitioner. If the results are the same, the medical practitioner confirms the conclusion and the application will proceed to the next step. If the medical practitioner conclusion does not match the generated result an alert will generated, to make the medical practitioner aware of the discrepancy. The medical practitioner can then choose to continue on with their original conclusion, or review the generated result, and be persuaded to change their original conclusion to concur with the application generated result. The next step in the program is to record the medical practitioner choice, and the computer generated choice to create statistics on how often medical practitioner or individual medical practitioners defer to the computer generated disposition, or how often the medical practitioner is more confident in their conclusion compared to the auto generated disposition. The program can then use all the previous medical data to determine if the patient is likely living with a chronic disposition, and is likely not following medical care advice. When this determination is made, the program can alert the medical care practitioners and more advanced care can be offered to that particular patient to ensure long term medical care for the chronic disease. Lastly using the previous information, stored information, and using the triage algorithm, the program can test and/or teach medical practitioners things about disposition conclusions in an environment where no patient welfare is at stake.

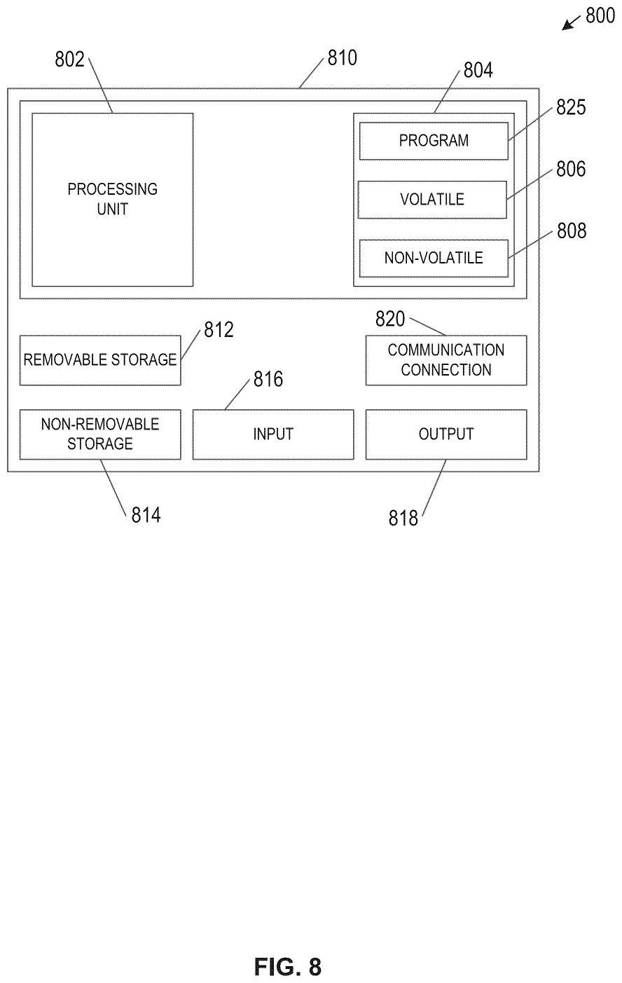

FIG. 1 is a flowchart illustrating a medical communication and triage assistant 100, according to an embodiment. The triage assistant 100 may be initialized when a patient begins speaking to a nurse or medical receptionist, may use one or more components within triage assistant 100 to collect and analyze medical information, and to generate medical data as described herein. While triage assistant 100 and computing device 800 are described herein with respect to a patient communicating with a nurse, the triage assistant 100 and computing device 800 may alternatively include a patient communicating with a medical receptionist, telehealth operator, text with prompts or other communication personnel. This communication may occur before a phone call is initiated between the patient and the medical practitioner, and the medical practitioner may interrupt the process at any time with a protocol or disposition. Once the medical data has been collected, analyzed, and generated, the medical data may be communicated to a medical services provider. While the components of medical communication and triage assistant 100 are described as steps, each of the components may be implemented in one or more computing devices or specialized circuits, such as using one more of the computing components described with respect to FIG. 8.

The medical communication and triage assistant 100 may use intelligent medical scripting and analysis to improve the efficiency and accuracy of capturing and analyzing medical data being entered by the nurse to analyzes the call. In an example, medical communication and triage assistant 100 may guide nurses to ask specific follow-up to improve the analysis of the patient symptoms and improve the prescribed disposition and care advice.

The medical communication and triage assistant 100 may include a history assistant 110. The history assistant 110 may be used to improve identifying, asking, and analyzing patient history, which may be crucial for making informed medical decisions and recommendations. The history assistant 110 supports nurses in gathering accurate and comprehensive patient information and documenting the information efficiently. In some example medical applications, up to 75% or more of medical diagnoses are made based on a patient's medical history. The history assistant 110 provides a standardized solution for a nurse to record accurate medical history in telehealth and other medical applications.

The history assistant 110 uses a history data collection process to ensure that all relevant patient medical details and patient symptom details are captured in a way that that is both machine readable and human readable. The history assistant 110 may be used to gather medical information, analyze the information, and generate mutually consistent machine readable and human readable medical information. The collected and generated medical data may be used to rule out a true emergency early during a communication session to avoid delays in care, where a true emergency may require a call to 911 or other emergency services. The collected and generated medical data may also be used to ensure the nurse has the details to determine the best protocol (e.g., decision tree) to use to evaluate the patient and select a disposition, and to generate and store data for subsequent analysis and automation.

The history assistant 110 may include an initial assessment. The initial assessment may be used for rapid and reliable determination of whether a 911 call or another emergency services request may be needed. In an example, this includes assessing airway, breathing, circulation, and neurological symptoms. This initial assessment may be used to reduce or minimize the time needed to initiate or rule out an emergency services request. The history assistant 110 may include a past medical history analysis. This past medical history analysis may facilitate identifying and recording chronic conditions, surgeries, allergies, medications, and other relevant patient data.

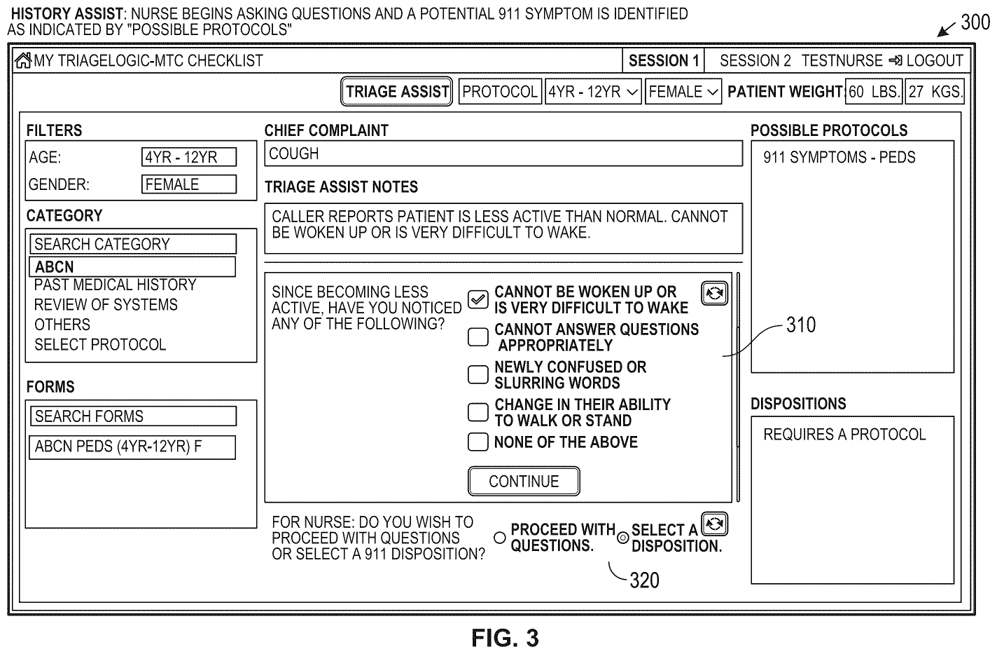

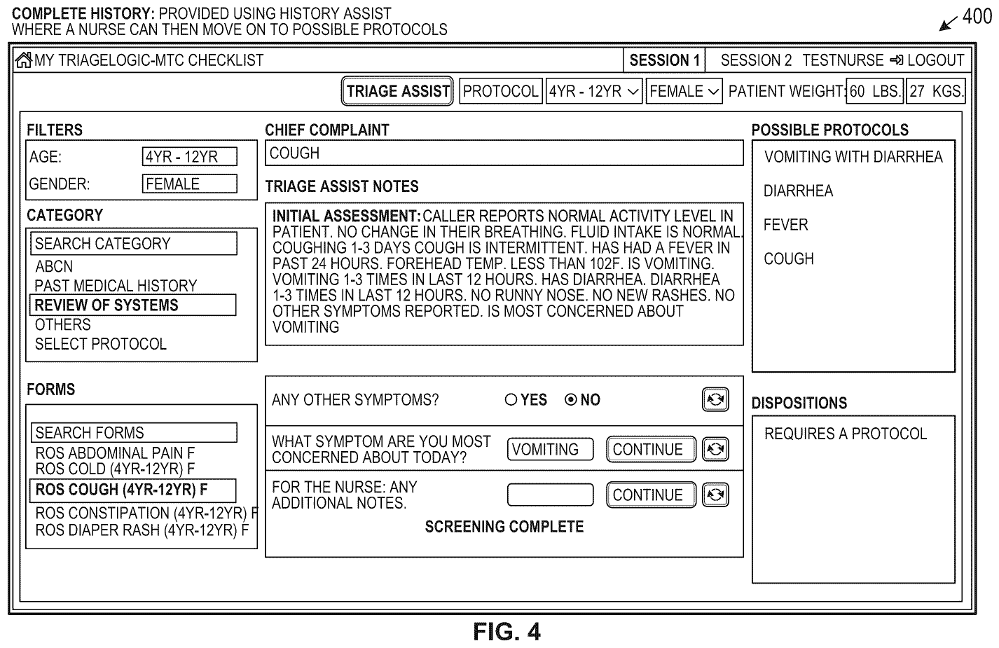

The history assistant 110 may include a symptom-based analysis. In an example, a graphical user interface (GUI) may present a number of patient symptoms for selection by the nurse, demonstrated in FIG. 3. Once a symptom is selected, additional details may be presented for selection, such as symptom duration, symptom severity, related symptoms, and other factors that assist in ensuring a complete and accurate characterization of symptoms and medical history. In an example, the GUI may present symptoms and additional details may be selected using check boxes, demonstrated in FIG. 4, and the history assistant 110 may use the selected check boxes to generate a machine readable list and a human readable description of symptoms and patient medical history. In an example, the human readable descriptions may include complete sentences, and the mapping of checked boxes to predetermined complete sentences may improve the use of the medical data by subsequent emergency services personnel or medical practitioners.

The history assistant 110 provides various technical advantages by gathering information in a standard format, analyzing that data, and generating both machine readable data and human readable data. This improved data collection and analysis provides standardization of input and review by nurses, medical practitioners, or emergency services personnel. This also improves speed and reduces errors in data collection and analysis, such as by avoiding freeform text entry or dictation. By generating both machine readable data and human readable data, this improves consistency between the recorded information and the medical information by nurses, medical practitioners, or emergency services personnel. By providing improved information collection accuracy and analysis, this generates recorded medical data that may be used for subsequent medical automation tasks and medical data analysis, such as to improve subsequent patient diagnosis.

The medical communication and triage assistant 100 may include a protocol assistant 120. The protocol assistant 120 may be used following the history assistant 110, such as when history assistant 110 determines that no emergency assistance call is required. The protocol assistant 120 may use a triage algorithm for medical analysis, such as for further patient symptom identification and analysis. The protocol assistant 120 may provide identification and recommendation of one or more medical triage protocols to be used based on the patient symptom identification and analysis. In an example, the history assistant 110 may be configured to gather or generate data that may be used by protocol assistant 120 to facilitate identification and recommendation of medical triage protocols.

The medical communication and triage assistant 100 may include a safety assistant 130. The safety assistant 130 may be used to reduce or eliminate the possibility that a nurse overlooks critical patient information. The safety assistant 130 may monitor the data collected by the history assistant 110 or the protocol assistant 120, and may generate an alert or recommendation if an urgent medical disposition might have been missed. This alert or recommendation may be used to confirm or reassess a medical triage decision made by the nurse.

The medical data gathered by the history assistant 110 may be used by the protocol assistant 120 to generate a medical disposition based on the patient symptoms, such as severity, duration, and other relevant medical data. In an example, the disposition may include recommending or initiating an emergency services request (e.g., 911), an urgent recommendation to be seen at an emergency room within four hours, a non-urgent recommendation to follow up in a medical practitioner office, or a home care recommendation.

Because medical data collected by the history assistant 110 is machine readable and correlated to the decision process in the protocol assistant 120, the safety assistant 130 can compare the disposition selected by the nurse to a disposition generated by the protocol assistant 120 based on the same data points and protocols. If there is a mismatch, the safety assistant 130 can alert the nurse and prompt them to reassess or confirm their decision. In an example, the disposition recommended by the nurse may be less urgent than a disposition made by protocol assistant 120, such as when a nurse recommends a non-urgent recommendation to follow up in a medical practitioner office but protocol assistant 120 recommends being seen at an emergency room within four hours. This may provide a safety check to improve patient care while reducing or minimizing the interference with a disposition made by the nurse or other medical communication personnel.

The triage assistant 100 may include a quality assurance assistant 140. The quality assurance assistant 140 may provide the ability to evaluate an expected or recommended medical protocol and disposition made during each medical request (e.g., telehealth call) and compare it against the selection made by the nurse to identify discrepancies. This may be used to provide an automated statistical analysis of the discrepancies, and may be used to generate machine readable and human readable statistical analysis data.

The evaluation provided by the quality assurance assistant 140 may include evaluating the nurse on protocol selection. In an example, the protocol selection may be evaluated based on whether the protocol was determined to be correct, incorrect, or acceptable (e.g., neither correct nor incorrect). The evaluation provided by the quality assurance assistant 140 may include evaluating the nurse on disposition selection. In an example, the disposition selection may be evaluated based on whether the disposition was determined to be correct or incorrect. The evaluation provided by the quality assurance assistant 140 may further include evaluating a sentiment analysis of the interaction with the patient. In an example, a text transcript of the interaction with the patient may be analyzed by a sentiment analysis device (e.g., a processor running an artificial intelligence (AI) model to a determine sentiment category or score). The quality assurance assistant 140 may provide one or more of the evaluations as statistics for human analysis, such as by providing a graphic (e.g., bar graph) or color-based indication of a specific nurse's protocol selection evaluation, disposition selection evaluation, or sentiment analysis evaluation.

In an example, the generated statistical analyses of the discrepancies may include such as a number, frequency, or magnitude of discrepancies. In another example, the generated statistical analyses of the discrepancies may include such as a number, frequency, or magnitude of the subset of decisions where the nurse changed their disposition based on a difference between their initial disposition and the disposition recommended by the protocol assistant 120.

This provides an improvement over systems that include a manual review of a small number of nurse decisions (e.g., 3-5 decisions per month). By using the history assistant 110, protocol assistant 120, and safety assistant 130, every medical assistance request (e.g., medical triage call) can be analyzed for potential outcomes and compared to the judgement of the nurse. This may be used to generate reports on variance from expected performance or deviations, and may be used to identify outliers and allow for further investigation.

The triage assistant 100 may include a medical communication assistant 150. Using the accurate medical data collected by history assistant 110, the medical communication assistant 150 may be used to generate and send a message to the patient who is seeking care. The patient may use the history assistant 110 to enter medical history information, which can be processed and provided to the medical practitioner. The medical communication assistant 150 may also be used to send a request to a patient with a home care disposition if they would like to observe their own symptoms for a specific period and then connect with a nurse or other medical practitioner if needed. This will provide improved options for home care and for reallocating urgent care resources to more critical patients based on medical priority.

The triage assistant 100 may include a chronic care assistant 160. By collecting specific medical data, the chronic care assistant 160 may be used to identify patients with chronic conditions who may not be compliant with their treatment plans (e.g., uncontrolled chronic conditions). The chronic care assistant 160 may be used to improve the health outcomes of patients with chronic conditions, such as by identifying patients with chronic conditions and providing secure communication of chronic medical data to primary care givers for enhanced medical management. This may also reduce health care costs for patients with chronic conditions or for the overall health system by providing improved health outcomes.

The triage assistant 100 may include a training assistant 170. The training assistant 170 may use advanced simulation technology to provide a safe and engaging environment for nurses to hone their medical triage skills, practice new techniques, and learn from real-life scenarios. This may provide improved patient diagnosis in a safe environment, such as by allowing nurses and other medical communication personnel to experience and diagnose hypothetical critical patient care scenarios to prepare for similar critical patient care scenarios.

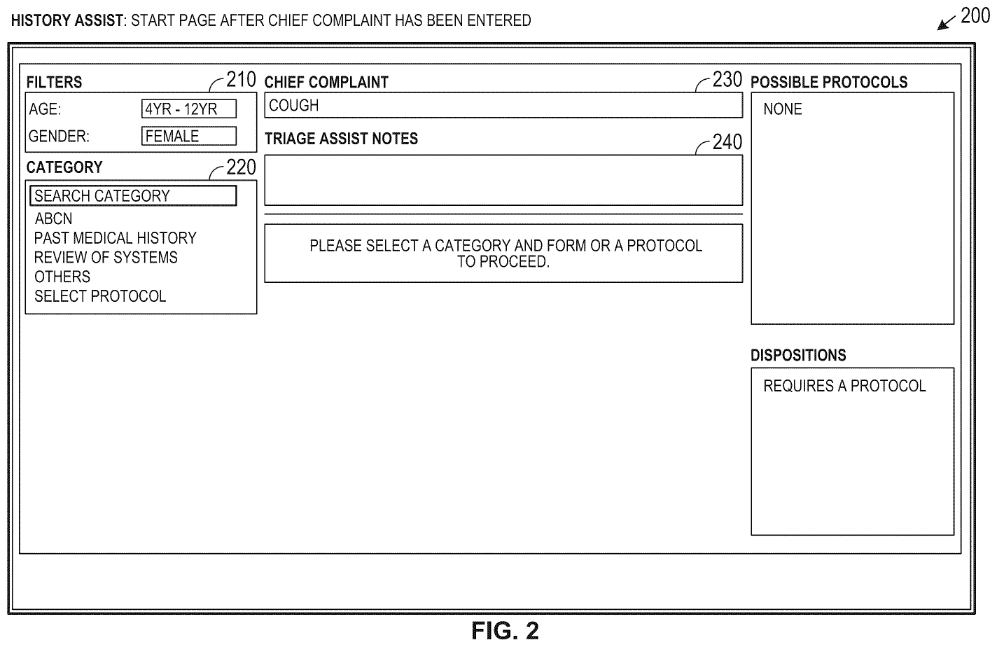

FIG. 2 is a GUI diagram illustrating an introductory display 200 of what a user could interact with, according to an embodiment. On the GUI, FIG. 2 shows the GUI containing a filter 210 and a category section to facilitate the input of patient history. The input options available for review by a medical practitioner in FIG. 2 are only age and gender, but in other embodiments the filter can be a wide variety of other medical data. For example, in a different embodiment, there could be a section in the patient history filter of the GUI that has a binary option of cardiac problems persisting in the family. Additionally, shown in FIG. 2, is an input screen for inputting a chief complaint 230 (e.g., a reason for the interaction with the application), and a separate area for another user input, to take notes 240 on the chief complaint where the application does not have a pre-established selection interface to enter the unique information from the patient. The notes can be relayed to a medical practitioner or anyone else who may interact with the patient. The notes could be used to ensure there were not any misinputs into the computer system where an incorrect disposition would be recommended due to the misinput. Notes could also be other information that the medical personal working on the patient would need to know, to help the patient but may not necessarily be considered history or symptoms. An example of this could be something like allergies, but any medical information that is not a symptom or history can be entered into the notes section of the GUI. In further embodiments of this application different medical practitioners would be able to update the notes section, to better find the appropriate caretaker to help the patient with the symptoms they would be experiencing.

FIG. 3 is a graphical user interface diagram 300, displaying a present symptom input, according to an embodiment. FIG. 3 shows the GUI displaying prompts o the medical practitioner to inquire about from the patient, to gather a full understanding of the symptoms of the patient. The prompts may include a comprehensive and complete set of check boxes to indicate present symptoms, but other embodiments may use other input methods to indicate to the application that a symptom is present. The intuitive design of the checkmarks helps the medical practitioner quickly enter present symptoms to allow the program to generate a response rapidly, in a potential situation where the patient would need immediate assistance. At the conclusion of the symptom input phase, the GUI shows an option to end symptom questioning and select a disposition.

At the top of the GUI, session details are displayed, including session identifiers and options for the nurse to log out, ensuring effective session management. The GUI provides a dynamic symptom checklist that adapts based on the chief complaint entered. For symptoms like decreased activity or responsiveness, the checklist updates to highlight these critical symptoms, prompting the nurse to ask further detailed questions to assess the condition's severity.

A prominent feature in this GUI is the emergency protocol activation section. If the symptoms entered meet the criteria for an emergency, such as difficulty waking the patient or other severe symptoms, the system highlights this with a visual cue (e.g., color change or flashing icon) and provides a direct option to initiate a 911 call. The GUI supports decision-making by offering choices to ‘Proceed with Questions’ or ‘Select a 911 Disposition.’ This allows the nurse to either continue gathering more information or to immediately escalate the situation based on the severity of the symptoms reported.

Interactive elements such as dropdown menus, checkboxes, and buttons are strategically placed to facilitate quick and accurate data entry, ensuring a logical flow of triage assessment, and minimizing the risk of oversight or error. The system is designed to provide real-time feedback and alerts based on the inputs. If a potentially life-threatening symptom is entered, the system immediately alerts the nurse and suggests appropriate emergency protocols, ensuring that critical conditions are not overlooked.

FIG. 4 is a graphical user interface diagram 400 illustrating a further progression of present symptom input 300, displaying other present symptom inputs, according to an embodiment. FIG. 4 shows additional methods of entering symptoms into the GUI, including typed responses. These type responses may be stored in computer-readable and human-readable data formats.

While graphical user interface diagram 400 shows text input, other forms of input may be possible. In an example, a GUI may display a digital portrayal of a human body (e.g., body outline, body avatar), and the medical practitioner may select a part of the human body where the symptom is persisting. The medical practitioner may also enter additional information about the symptoms. Data received from the digital portrayal or the medical practitioner may be converted and stored in computer-readable and human-readable data formats.

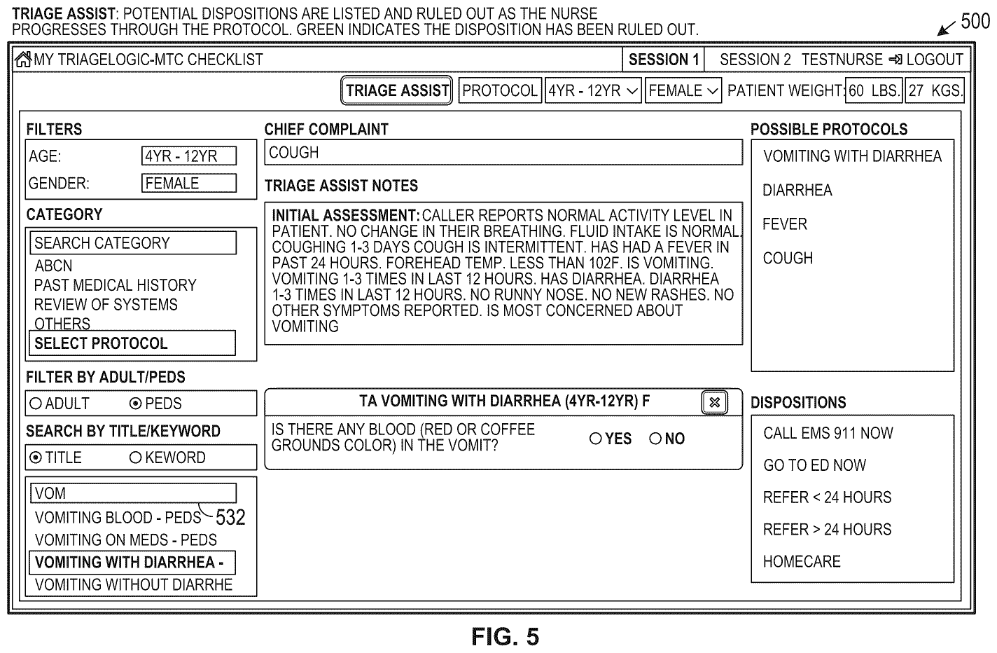

FIG. 5 is a graphical user interface diagram 500, displaying an emphasis on rejected dispositions according to an embodiment. This GUI shows possible protocol selections, and potential disposition recommendations for a medical practitioner to select. This GUI further shows how possible dispositions can be emphasized in diverse ways to indicate different urgencies, or possibilities of likelihood that the disposition is correct based on computer evaluations. Furthermore, the GUI shows different options to input and search for symptoms on the GUI.

The GUI further provides a display of rejected dispositions. This aspect of the GUI allows medical practitioners to see which potential dispositions have been considered and ruled out during the triage process. This visual representation helps in understanding the decision-making pathway and ensures that various options are thoroughly evaluated before arriving at a final decision.

The GUI provides options to input and search for symptoms, which enhances the nurse's ability to navigate through vast medical data efficiently. This feature likely includes search functionalities that allow the practitioner to quickly locate specific symptoms or related medical conditions, streamlining the process of symptom checking and protocol selection.

The GUI may be configured to support critical decision-making in medical triage by visually distinguishing between different medical dispositions based on their urgency and appropriateness. The interface may use color coding, icons, or other graphical elements to indicate the urgency level of each disposition, such as using red to denote emergency actions or green for less urgent care steps. This visual guidance aids practitioners in quickly assessing the patient's needs and deciding on the most appropriate medical response.

The GUI's ability to display both rejected and recommended dispositions provides a comprehensive overview of the decision-making process, ensuring that practitioners are aware of all considerations taken into account. This feature enhances the safety and accuracy of the medical triage, and serves as a learning tool for practitioners to understand the rationale behind each decision.

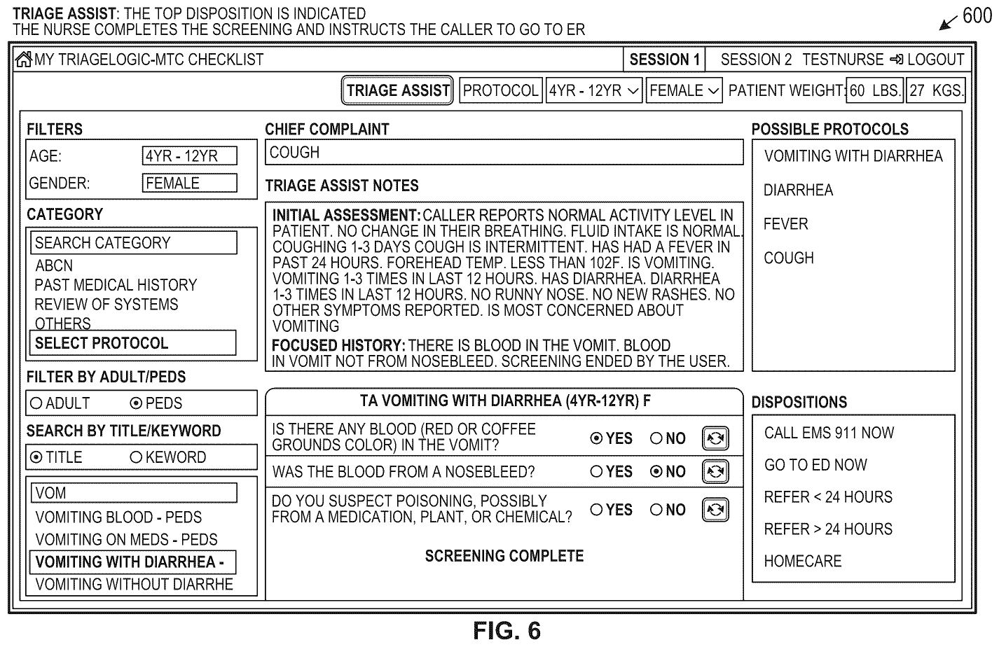

FIG. 6 is a graphical user interface diagram 600, displaying an emphasis on likely dispositions according to an embodiment. Similar to FIG. 5, FIG. 6 shows different emphasis ways to indicate different urgencies, or possibilities based on computer recommendations.

As shown in FIG. 6, the GUI provides an indication for emergency medical services (EMS). This includes a clear directive for the patient to go to the emergency department immediately, which may be emphasized visually in the GUI. This feature provides improved responsiveness in situations where immediate medical intervention is necessary, and it helps ensure that there is no delay in the patient receiving the required care.

The GUI in FIG. 6 is designed to facilitate quick and decisive action in emergency situations. The interface may use colors or distinct visual cues to highlight the urgency of the situation, guiding the nurse to advise the patient accurately and promptly. Additionally, the GUI provides a focused history summary section where critical symptoms that led to the emergency decision. This improves the decision-making process and improves consideration of relevant information making such a critical recommendation.

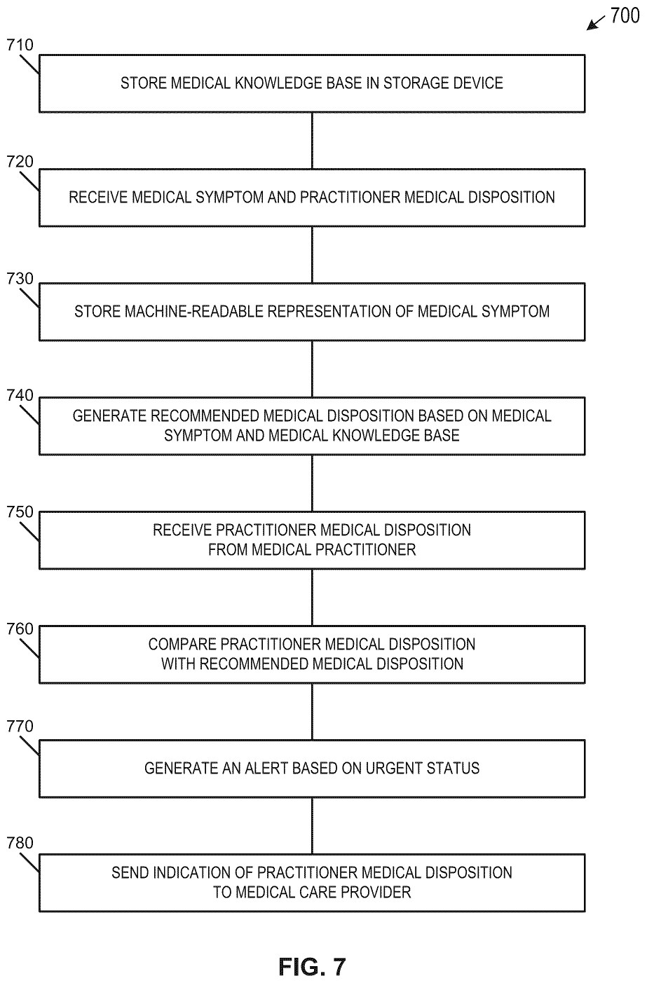

FIG. 7 is a flowchart illustrating a medical communication and triage assistant method 700, according to an embodiment. Method 700 includes storing 710 a medical knowledge base in a storage device and receiving 720 at least one medical symptom associated with a patient and a practitioner medical disposition provided by a medical practitioner via an input device. Method 700 includes storing 730 a machine-readable representation of the at least one medical symptom in the storage device. Method 700 includes generating 740 a recommended medical disposition based on the machine-readable representation of the at least one medical symptom and the medical knowledge base. Method 700 includes receiving 750 a practitioner medical disposition from the medical practitioner and comparing 760 this disposition with the recommended medical disposition. Method 700 includes generating 770 an alert when the practitioner medical disposition has an associated non-urgent status and the recommended medical disposition has an associated urgent status. Method 700 includes sending 780 an indication of the practitioner medical disposition to a medical care provider.

Method 700 may further include storing a human-readable representation of the at least one medical symptom alongside the machine-readable representation in the storage device. The human-readable representation may be sent to the medical care provider, enhancing communication and clarity of medical data. The medical care provider may include a physician, an urgent care facility, an emergency room facility, a clinic, a hospital, or similar medical care provider.

Method 700 may further include prompting a medical practitioner with a plurality of medical history questions to be asked of the patient. The method collects a plurality of medical history responses based on these questions and stores these responses in both a human-readable response format and a computer-readable response format for subsequent review and quality assurance analysis.

Method 700 may further include determining a recommended ongoing medical care program based on the patient's medical history and the collected medical history responses. Method 700 may further include identifying a chronic condition based on the medical history responses. This may involves prompting the medical practitioner with specific questions about the chronic condition, determining if the patient has been following a chronic condition care plan, and generating an alert for the medical practitioner if the patient has not been following the plan.

Method 700 may further include generating a hypothetical patient with hypothetical symptoms for the training of medical practitioners. This may include generating hypothetical symptom questions and a medical recommendation, prompting the training medical practitioner with these questions, receiving a hypothetical practitioner recommendation, and generating a training summary based on identified differences between the recommendations.

Method 700 may further include tracking historical comparisons between historical practitioner medical dispositions and historical recommended medical dispositions. This may be used for analyzing trends and improving medical dispositions over time. Method 700 may further include storing medical practitioner disposition statistics and generating a historical report based on these statistics, which aids in the assessment and improvement of medical practice standards.

Method 700 may further include receiving an updated mapping between a set of patient symptoms and a set of medical dispositions, and generating future medical dispositions based on this updated mapping. Method 700 may further include generating a plurality of medical treatment protocols based on both the practitioner medical disposition and the recommended medical disposition. This may improve the ability for the triage system to remain current and effective.

Method 700 may further include eliminating a set of medical conditions based on the at least one medical symptom, identifying a plurality of potential medical conditions based on the at least one medical symptom, and identifying the most likely medical condition, thereby refining the diagnostic process. This may include identifying a medical symptom clarifying prompt for the medical practitioner to ask the patient, receiving a clarifying prompt answer, and identifying a revised likely medical condition based on the clarifying prompt answer and the plurality of potential medical conditions, enhancing the accuracy of diagnosis.

Method 700 may further include determining the patient needs urgent medical attention prior to determining a non-urgent disposition. This provides improve prioritization of patient safety and immediate care needs. Method 700 may further include generating the recommended medical disposition based on at least one of symptom severity or symptom duration, providing a nuanced approach to determining medical dispositions.

Method 700 may further include receiving, within a graphical user interface, at least one patient medical symptom provided by the patient, generating an initial medical disposition based on the at least one patient medical symptom, and generating an indication for the patient to contact a medical practitioner for further medical advice, enhancing patient engagement and proactive care. Method 700 may further include receiving, within the graphical user interface, at least one patient historical medical symptom provided by the patient, identifying a persistence of symptoms based on the at least one patient medical symptom and the at least one patient historical medical symptom, and generating an indication for the patient to contact the medical practitioner based on the persistence of symptoms, ensuring continuous monitoring and care.

FIG. 8 is a block diagram of a computing device 800, according to an embodiment. The performance of one or more components within computing device 800 may be improved by including one or more of the circuits or circuitry methods described herein. Computing device 800 may include a system for relaying a medical message, according to an embodiment. In one embodiment, multiple such computer systems are used in a distributed network to implement multiple components in a transaction-based environment. An object-oriented, service-oriented, or other architecture may be used to implement such functions and communicate between the multiple systems and components. In some embodiments, the computing device of FIG. 8 is an example of a client device that may invoke methods described herein over a network. In some embodiments, the computing device of FIG. 8 is an example of one or more of the personal computer, smartphone, tablet, or various servers.

One example computing device in the form of a computer 810, may include a processing unit 802, memory 804, removable storage 812, and non-removable storage 814. Although the example computing device is illustrated and described as computer 810, the computing device may be in different forms in different embodiments. For example, the computing device may instead be a smartphone, a tablet, or other computing device including the same or similar elements as illustrated and described with regard to FIG. 8. Further, although the various data storage elements are illustrated as part of the computer 810, the storage may include cloud-based storage accessible via a network, such as the Internet.

Returning to the computer 810, memory 804 may include volatile memory 806 and non-volatile memory 808. Computer 810 may include or have access to a computing environment that includes a variety of computer-readable media, such as volatile memory 806 and non-volatile memory 808, removable storage 812 and non-removable storage 814. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions. Computer 810 may include or have access to a computing environment that includes input 816, output 818, and a communication connection 820. The input 816 may include one or more of a touchscreen, touchpad, mouse, keyboard, camera, and other input devices. The input 816 may include a navigation sensor input, such as a GNSS receiver, a SOP receiver, an inertial sensor (e.g., accelerometers, gyroscopes), a local ranging sensor (e.g., LIDAR), an optical sensor (e.g., cameras), or other sensors. The computer may operate in a networked environment using a communication connection 820 to connect to one or more remote computers, such as database servers, web servers, and another computing device. An example remote computer may include a personal computer (PC), server, router, network PC, a peer device or other common network node, or the like. The communication connection 820 may be a network interface device such as one or both of an Ethernet card and a wireless card or circuit that may be connected to a network. The network may include one or more of a Local Area Network (LAN), a Wide Area Network (WAN), the Internet, and other networks.

Computer-readable instructions stored on a computer-readable medium are executable by the processing unit 802 of the computer 810. A hard drive (magnetic disk or solid state), CD-ROM, and RAM are some examples of articles including a non-transitory computer-readable medium. For example, various computer programs 825 or apps, such as one or more applications and modules implementing one or more of the methods illustrated and described herein or an app or application that executes on a mobile device or is accessible via a web browser, may be stored on a non-transitory computer-readable medium.

The apparatuses and methods described above may include or be included in high-speed computers, communication and signal processing circuitry, single-processor module or multi-processor modules, single embedded processors or multiple embedded processors, multi-core processors, message information switches, and application-specific modules including multilayer or multi-chip modules. Such apparatuses may further be included as sub-components within a variety of other apparatuses (e.g., electronic systems), such as televisions, cellular telephones, personal computers (e.g., laptop computers, desktop computers, handheld computers, etc.), tablets (e.g., tablet computers), workstations, radios, video players, audio players (e.g., MP3 (Motion Picture Experts Group, Audio Layer 3) players), vehicles, medical devices (e.g., heart monitors, blood pressure monitors, etc.), set top boxes, and others.

In the detailed description and the claims, the term “on” used with respect to two or more elements (e.g., materials), one “on” the other, means at least some contact between the elements (e.g., between the materials). The term “over” means the elements (e.g., materials) are in close proximity, but possibly with one or more additional intervening elements (e.g., materials) such that contact is possible but not required. Neither “on” nor “over” implies any directionality as used herein unless stated as such.

In the detailed description and the claims, a list of items joined by the term “at least one of” may mean any combination of the listed items. For example, if items A and B are listed, then the phrase “at least one of A and B” means A only; B only; or A and B. In another example, if items A, B, and C are listed, then the phrase “at least one of A, B and C” means A only; B only; C only; A and B (excluding C); A and C (excluding B); B and C (excluding A); or all of A, B, and C. Item A may include a single element or multiple elements. Item B may include a single element or multiple elements. Item C may include a single element or multiple elements.

In the detailed description and the claims, a list of items joined by the term “one of” may mean only one of the list items. For example, if items A and B are listed, then the phrase “one of A and B” means A only (excluding B), or B only (excluding A). In another example, if items A, B, and C are listed, then the phrase “one of A, B and C” means A only; B only; or C only. Item A may include a single element or multiple elements. Item B may include a single element or multiple elements. Item C may include a single element or multiple elements.

### Additional Notes and Examples

Example 1 is a method for medical triage in a remote-health system, comprising: storing a medical knowledge base in a storage device; receiving at least one medical symptom associated with a patient and a practitioner medical disposition provided by a medical practitioner via an input device; storing a machine-readable representation of the at least one medical symptom in the storage device; generating a recommended medical disposition based on the machine-readable representation of the at least one medical symptom and the medical knowledge base; receiving a practitioner medical disposition from the medical practitioner; comparing the practitioner medical disposition with the recommended medical disposition; generating an alert when the practitioner medical disposition has an associated non-urgent status and the recommended medical disposition has an associated urgent status; sending an indication of the practitioner medical disposition to a medical care provider.

In Example 2, the subject matter of Example 1 includes storing a human-readable representation of the at least one medical symptom and the machine-readable representation of the at least one medical symptom in the storage device; and sending the human-readable representation to the medical care provider.

In Example 3, the subject matter of Examples 1-2 includes wherein the medical care provider includes at least one of a physician, an urgent care facility, an emergency room facility, a clinic, or a hospital.

In Example 4, the subject matter of Examples 1-3 includes prompting a medical practitioner with a plurality of medical history questions to be asked of the patient; collecting a plurality of medical history responses based on the plurality of medical history questions; and storing the plurality of medical history responses in a human-readable response format and a computer-readable response format for subsequent review and analysis.

In Example 5, the subject matter of Example 4 includes determining a recommended ongoing medical care program based on the patient medical history and the plurality of medical history responses.

In Example 6, the subject matter of Examples 4-5 includes identifying a chronic condition based on the plurality of medical history responses; prompting the medical practitioner with chronic medical condition questions; determining the patient has not been following a chronic condition care plan; and generating an alert indicating non-compliance with the care plan.

In Example 7, the subject matter of Examples 1-6 includes generating a hypothetical patient with hypothetical symptoms for training a medical practitioner; generating hypothetical symptom questions and a medical recommendation; prompting the training medical practitioner with the hypothetical symptom questions; receiving a hypothetical practitioner recommendation; and generating a training summary based on identified differences between the recommendations.

In Example 8, the subject matter of Examples 1-7 includes tracking historical comparisons between historical practitioner medical dispositions and historical recommended medical dispositions.

In Example 9, the subject matter of Example 8 includes storing medical practitioner disposition statistics; and generating a historical report based on these statistics.

In Example 10, the subject matter of Examples 8-9 includes receiving an updated mapping between a set of patient symptoms and a set of medical dispositions; and generating future medical dispositions based on the updated mapping.

In Example 11, the subject matter of Examples 8-10 includes generating a plurality of medical treatment protocols based on the practitioner medical disposition and the recommended medical disposition.

In Example 12, the subject matter of Examples 1-11 includes eliminating a set of medical conditions based on the at least one medical symptom; identifying a plurality of potential medical conditions based on the at least one medical symptom; and identifying the most likely medical condition.

In Example 13, the subject matter of Example 12 includes identifying a medical symptom clarifying prompt for the medical practitioner to ask the patient; receiving a clarifying prompt answer; and identifying a revised likely medical condition based on the clarifying prompt answer.

In Example 14, the subject matter of Examples 1-13 includes determining the patient needs urgent medical attention prior to determining a non-urgent disposition.

In Example 15, the subject matter of Examples 5-14 includes generating the recommended medical disposition based on at least one of symptom severity or symptom duration.

In Example 16, the subject matter of Examples 1-15 includes receiving, within a graphical user interface, at least one patient medical symptom provided by the patient; generating an initial medical disposition based on the at least one patient medical symptom; and generating an indication for the patient to contact a medical practitioner for further medical advice.

In Example 17, the subject matter of Example 16 includes receiving, within the graphical user interface, at least one patient historical medical symptom provided by the patient; identifying a persistence of symptoms based on the at least one patient medical symptom and the at least one patient historical medical symptom; and generating an indication for the patient to contact the medical practitioner based on the persistence of symptoms.

Example 18 is a remote-health system for medical triage, the system comprising: a storage device including a medical knowledge base; an input device configured to receive at least one medical symptom associated with a patient and a practitioner medical disposition provided by a medical practitioner; and a processing circuitry configured to: store a machine-readable representation of the at least one medical symptom in the storage device; generate a recommended medical disposition based on the machine-readable representation of the at least one medical symptom and the medical knowledge base; receive a practitioner medical disposition from the medical practitioner; compare the practitioner medical disposition with the recommended medical disposition; generate an alert when the practitioner medical disposition has an associated non-urgent status and the recommended medical disposition has an associated urgent status; and send an indication of the practitioner medical disposition to a medical care provider.

In Example 19, the subject matter of Example 18 includes the processing circuitry further to: store a human-readable representation of the at least one medical symptom and the machine-readable representation of the least one medical symptom in the storage device; and send the human-readable representation to the medical care provider.

In Example 20, the subject matter of Examples 18-19 includes wherein the medical care provider includes at least one of a physician, an urgent care facility, an emergency room facility, a clinic, or a hospital.

In Example 21, the subject matter of Examples 18-20 includes wherein the processing circuitry is further configured to: prompt a medical practitioner with a plurality of medical history questions to be asked of the patient; collect a plurality of medical history responses based on the plurality of medical history questions; store the plurality of medical history responses in a human-readable response format for subsequent review by a physician; and store the plurality of medical history responses in a computer-readable response format for subsequent quality assurance analysis.

In Example 22, the subject matter of Example 21 includes wherein the processing circuitry is further configured to determine a recommended ongoing medical care program based on the patient medical history and the plurality of medical history responses.

In Example 23, the subject matter of Examples 21-22 includes the processing circuitry is further configured to: identify a chronic condition based on the plurality of medical history responses; prompt the medical practitioner with a plurality of chronic medical condition questions to be asked of the patient; determine the patient has not been following a chronic condition care plan; and generate an alert for the medical practitioner indicating the patient has not been following a chronic condition care plan.

In Example 24, the subject matter of Examples 18-23 includes wherein the processing circuitry is further configured to: generate a hypothetical patient with a plurality of hypothetical symptoms, the hypothetical patient for training of a training medical practitioner; generate a plurality of hypothetical symptom questions and a hypothetical medical recommendation based on the plurality of hypothetical symptoms; prompt the training medical practitioner with the plurality of hypothetical symptom questions; receive a hypothetical practitioner recommendation from the training medical practitioner; and generate a training summary based on a plurality of identified differences between the hypothetical practitioner recommendation and the hypothetical medical recommendation.

In Example 25, the subject matter of Examples 18-24 includes wherein the processing circuitry is further configured to track a plurality of historical comparisons between a plurality of historical practitioner medical dispositions and a plurality of historical recommended medical dispositions.

In Example 26, the subject matter of Example 25 includes the processing circuitry is further configured to: store a plurality of medical practitioner disposition statistics; and generate a historical report based on the plurality of medical practitioner disposition statistics.

In Example 27, the subject matter of Examples 25-26 includes wherein the processing circuitry is further configured to: receive an updated mapping between a set of patient symptoms and a set of medical dispositions; and generate future medical dispositions based on the updated mapping.

In Example 28, the subject matter of Examples 25-27 includes wherein the processing circuitry is further configured to generate a plurality of medical treatment protocols based on the practitioner medical disposition and the recommended medical disposition.

In Example 29, the subject matter of Examples 18-28 includes wherein the processing circuitry is further configured to: eliminate a set of medical conditions based on the at least one medical symptom; identify a plurality of potential medical conditions based on the at least one medical symptom; and identify a most likely medical condition based on the at least one medical symptom.

In Example 30, the subject matter of Example 29 includes the processing circuitry is further configured to: identify a medical symptom clarifying prompt for the medical practitioner to ask the patient, the medical symptom clarifying prompt to narrow a number of medical conditions among the plurality of potential medical conditions; receive a clarifying prompt answer from the medical practitioner; and identify a revised likely medical condition based on the clarifying prompt answer and the plurality of potential medical conditions.

In Example 31, the subject matter of Examples 18-30 includes wherein the processing circuitry is further configured to determine the patient needs urgent medical attention prior to determining a non-urgent disposition.

In Example 32, the subject matter of Examples 22-31 includes wherein the processing circuitry is further configured to generate the recommended medical disposition based on at least one of a symptom severity or a symptom duration.

In Example 33, the subject matter of Examples 18-32 includes wherein the processing circuitry is further configured to: receive, within a graphical user interface, at least one patient medical symptom provided by the patient; generate an initial medical disposition based on the at least one patient medical symptom; and generate an indication for the patient to contact a medical practitioner for further medical advice based on the initial medical disposition.

In Example 34, the subject matter of Example 33 includes the processing circuitry is further configured to: receive, within the graphical user interface, at least one patient historical medical symptom provided by the patient; identify a persistence of symptoms based on the at least one patient medical symptom and the at least one patient historical medical symptom; and generate an indication for the patient to contact the medical practitioner based on the persistence of symptoms.

Example 35 is a machine-readable storage medium comprising instructions that, when executed by processing circuitry of an electronic device, cause the processing circuitry to: store a medical knowledge base in a storage device; receive at least one medical symptom associated with a patient and a practitioner medical disposition provided by a medical practitioner via an input device; store a machine-readable representation of the at least one medical symptom in the storage device; generate a recommended medical disposition based on the machine-readable representation of the at least one medical symptom and the medical knowledge base; receive a practitioner medical disposition from the medical practitioner; compare the practitioner medical disposition with the recommended medical disposition; generate an alert when the practitioner medical disposition has an associated non-urgent status and the recommended medical disposition has an associated urgent status; send an indication of the practitioner medical disposition to a medical care provider.

In Example 36, the subject matter of Example 35 includes the instructions further causing the processing circuitry to: store a human-readable representation of the at least one medical symptom and the machine-readable representation of the at least one medical symptom in the storage device; and send the human-readable representation to the medical care provider.

In Example 37, the subject matter of Examples 35-36 includes wherein the medical care provider includes at least one of a physician, an urgent care facility, an emergency room facility, a clinic, or a hospital.

In Example 38, the subject matter of Examples 35-37 includes the instructions further causing the processing circuitry to: prompt a medical practitioner with a plurality of medical history questions to be asked of the patient; collect a plurality of medical history responses based on the plurality of medical history questions store the plurality of medical history responses in a human-readable response format and a computer-readable response format for subsequent review and analysis.

In Example 39, the subject matter of Example 38 includes the instructions further causing the processing circuitry to determine a recommended ongoing medical care program based on the patient medical history and the plurality of medical history responses.

In Example 40, the subject matter of Examples 38-39 includes the instructions further causing the processing circuitry to: identify a chronic condition based on the plurality of medical history responses; prompt the medical practitioner with chronic medical condition questions; determine the patient has not been following a chronic condition care plan; and generating an alert indicating non-compliance with the care plan.

In Example 41, the subject matter of Examples 35-40 includes the instructions further causing the processing circuitry to: generate a hypothetical patient with hypothetical symptoms for training a medical practitioner; generate hypothetical symptom questions and a medical recommendation; prompt the training medical practitioner with the hypothetical symptom questions; receive a hypothetical practitioner recommendation; and generate a training summary based on identified differences between the recommendations.

In Example 42, the subject matter of Examples 35-41 includes the instructions further causing the processing circuitry to track historical comparisons between historical practitioner medical dispositions and historical recommended medical dispositions.

In Example 43, the subject matter of Example 42 includes the instructions further causing the processing circuitry to store medical practitioner disposition statistics and generating a historical report based on these statistics.

In Example 44, the subject matter of Examples 42-43 includes the instructions further causing the processing circuitry to: receive an updated mapping between a set of patient symptoms and a set of medical dispositions; and generate future medical dispositions based on the updated mapping.

In Example 45, the subject matter of Examples 42-44 includes the instructions further causing the processing circuitry to generate a plurality of medical treatment protocols based on the practitioner medical disposition and the recommended medical disposition.

In Example 46, the subject matter of Examples 35-45 includes the instructions further causing the processing circuitry to: eliminate a set of medical conditions based on the at least one medical symptom; identify a plurality of potential medical conditions based on the at least one medical symptom; and identify the most likely medical condition.

In Example 47, the subject matter of Example 46 includes the instructions further causing the processing circuitry to: identify a medical symptom clarifying prompt for the medical practitioner to ask the patient; receive a clarifying prompt answer; and identify a revised likely medical condition based on the clarifying prompt answer.

In Example 48, the subject matter of Examples 35-47 includes the instructions further causing the processing circuitry to determine the patient needs urgent medical attention prior to determining a non-urgent disposition.

In Example 49, the subject matter of Examples 39-48 includes the instructions further causing the processing circuitry to generate the recommended medical disposition based on at least one of symptom severity or symptom duration.

In Example 50, the subject matter of Examples 35-49 includes the instructions further causing the processing circuitry to: receive, within a graphical user interface, at least one patient medical symptom provided by the patient; generate an initial medical disposition based on the at least one patient medical symptom; and generate an indication for the patient to contact a medical practitioner for further medical advice.

In Example 51, the subject matter of Example 50 includes the instructions further causing the processing circuitry to: receive, within the graphical user interface, at least one patient historical medical symptom provided by the patient; identify a persistence of symptoms based on the at least one patient medical symptom and the at least one patient historical medical symptom; and generate an indication for the patient to contact the medical practitioner based on the persistence of symptoms.

Example 52 is at least one machine-readable medium including instructions that, when executed by processing circuitry, cause the processing circuitry to perform operations to implement of any of Examples 1-51.

Example 53 is an apparatus comprising means to implement of any of Examples 1-51.

Example 54 is a system to implement of any of Examples 1-51.

Example 55 is a method to implement of any of Examples 1-51.

The subject matter of any Examples above may be combined in any combination.

The above description and the drawings illustrate some embodiments of the inventive subject matter to enable those skilled in the art to practice the embodiments of the inventive subject matter. Other embodiments may incorporate structural, logical, electrical, process, and other changes. Examples merely typify possible variations. Portions and features of some embodiments may be included in, or substituted for, those of others. Many other embodiments will be apparent to those of skill in the art upon reading and understanding the above description.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A method for medical triage in a remote-health system, comprising:
storing a medical knowledge base in a storage device;
receiving at least one medical symptom associated with a patient and a practitioner medical disposition provided by a medical practitioner via an input device;
storing a machine-readable representation of the at least one medical symptom in the storage device;
generating a recommended medical disposition based on the machine-readable representation of the at least one medical symptom and the medical knowledge base;
receiving a practitioner medical disposition from the medical practitioner;
comparing the practitioner medical disposition with the recommended medical disposition;
generating an alert when the practitioner medical disposition has an associated non-urgent status and the recommended medical disposition has an associated urgent status;
sending an indication of the practitioner medical disposition to a medical care provider.

2. The method of claim 1, further comprising:
storing a human-readable representation of the at least one medical symptom and the machine-readable representation of the at least one medical symptom in the storage device; and
sending the human-readable representation to the medical care provider.

3. The method of claim 1, further comprising:
prompting a medical practitioner with a plurality of medical history questions to be asked of the patient;
collecting a plurality of medical history responses based on the plurality of medical history questions; and
storing the plurality of medical history responses in a human-readable response format and a computer-readable response format for subsequent review and analysis.

4. The method of claim 3, further comprising:
identifying a chronic condition based on the plurality of medical history responses;
prompting the medical practitioner with chronic medical condition questions;
determining the patient has not been following a chronic condition care plan; and
generating an alert indicating non-compliance with the care plan.

5. The method of claim 1, further comprising:
generating a hypothetical patient with hypothetical symptoms for training a medical practitioner;
generating hypothetical symptom questions and a medical recommendation;
prompting the training medical practitioner with the hypothetical symptom questions;
receiving a hypothetical practitioner recommendation; and
generating a training summary based on identified differences between the recommendations.

6. The method of claim 1, further comprising tracking historical comparisons between historical practitioner medical dispositions and historical recommended medical dispositions.

7. The method of claim 6, further comprising:
storing medical practitioner disposition statistics; and
generating a historical report based on these statistics.

8. The method of claim 6, further comprising:
receiving an updated mapping between a set of patient symptoms and a set of medical dispositions; and
generating future medical dispositions based on the updated mapping.

9. The method of claim 1, further comprising:
eliminating a set of medical conditions based on the at least one medical symptom;
identifying a plurality of potential medical conditions based on the at least one medical symptom; and
identifying the most likely medical condition.

10. A remote-health system for medical triage, the system comprising:
a storage device including a medical knowledge base;
an input device configured to receive at least one medical symptom associated with a patient and a practitioner medical disposition provided by a medical practitioner; and
a processing circuitry configured to:
store a machine-readable representation of the at least one medical symptom in the storage device;
generate a recommended medical disposition based on the machine-readable representation of the at least one medical symptom and the medical knowledge base;
receive a practitioner medical disposition from the medical practitioner;
compare the practitioner medical disposition with the recommended medical disposition;
generate an alert when the practitioner medical disposition has an associated non-urgent status and the recommended medical disposition has an associated urgent status; and
send an indication of the practitioner medical disposition to a medical care provider.

11. The system of claim 10, the processing circuitry further to:
store a human-readable representation of the at least one medical symptom and the machine-readable representation of the least one medical symptom in the storage device; and
send the human-readable representation to the medical care provider.

12. The system of claim 10, wherein the processing circuitry is further configured to: prompt a medical practitioner with a plurality of medical history questions to be asked of the patient;
collect a plurality of medical history responses based on the plurality of medical history questions;
store the plurality of medical history responses in a human-readable response format for subsequent review by a physician; and
store the plurality of medical history responses in a computer-readable response format for subsequent quality assurance analysis.

13. The system of claim 12, wherein the processing circuitry is further configured to determine a recommended ongoing medical care program based on the patient medical history and the plurality of medical history responses.

14. The system of claim 12, the processing circuitry is further configured to:
identify a chronic condition based on the plurality of medical history responses;
prompt the medical practitioner with a plurality of chronic medical condition questions to be asked of the patient;
determine the patient has not been following a chronic condition care plan; and
generate an alert for the medical practitioner indicating the patient has not been following a chronic condition care plan.

15. The system of claim 10, wherein the processing circuitry is further configured to track a plurality of historical comparisons between a plurality of historical practitioner medical dispositions and a plurality of historical recommended medical dispositions.

16. The system of claim 15, the processing circuitry is further configured to:
store a plurality of medical practitioner disposition statistics; and
generate a historical report based on the plurality of medical practitioner disposition statistics.

17. The system of claim 15, wherein the processing circuitry is further configured to:
receive an updated mapping between a set of patient symptoms and a set of medical dispositions; and
generate future medical dispositions based on the updated mapping.

18. The system of claim 10, wherein the processing circuitry is further configured to:
eliminate a set of medical conditions based on the at least one medical symptom;
identify a plurality of potential medical conditions based on the at least one medical symptom; and
identify a most likely medical condition based on the at least one medical symptom.

19. The system of claim 18, the processing circuitry is further configured to:
identify a medical symptom clarifying prompt for the medical practitioner to ask the patient, the medical symptom clarifying prompt to narrow a number of medical conditions among the plurality of potential medical conditions;
receive a clarifying prompt answer from the medical practitioner; and
identify a revised likely medical condition based on the clarifying prompt answer and the plurality of potential medical conditions.

20. A machine-readable storage medium comprising instructions that, when executed by processing circuitry of an electronic device, cause the processing circuitry to:
store a medical knowledge base in a storage device;
receive at least one medical symptom associated with a patient and a practitioner medical disposition provided by a medical practitioner via an input device;
store a machine-readable representation of the at least one medical symptom in the storage device;
generate a recommended medical disposition based on the machine-readable representation of the at least one medical symptom and the medical knowledge base;
receive a practitioner medical disposition from the medical practitioner;
compare the practitioner medical disposition with the recommended medical disposition;
generate an alert when the practitioner medical disposition has an associated non-urgent status and the recommended medical disposition has an associated urgent status;
send an indication of the practitioner medical disposition to a medical care provider.
